(19) United States
(12) Patent Application Publication
(10) Pub. No.: US 2007/0244411 A1
(43) Pub. Date: Oct. 18, 2007

(54) METHOD AND SYSTEM FOR MONITORING INTRACRANIAL PRESSURE

(75) Inventors: Patricia S. Jeng, Mahomet, IL (US); Susan Voss, Northampton, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Mimosa Acoustics, Inc., Champaign, IL (US)

(21) Appl. No.: 11/696,037

(22) Filed: Apr. 3, 2007

Related U.S. Application Data
(60) Provisional application No. 60/788,984, filed on Apr. 3, 2006.

(51) Int. Cl. A61B 5/00

(52) U.S. Cl. 600/561

(57) ABSTRACT

Method and system for monitoring intracranial pressure. According to an embodiment, the present invention provides a method for monitoring intracranial pressure (ICP) for at least one patient. The method includes positioning an ear probe into an ear canal. The method further includes measuring a first acoustic reflectance using the ear probe at a first time. The first acoustic reflectance is associated with the ear canal as a function of an incident pressure and an acoustic frequency. The method additionally includes processing information associated with the first acoustic reflectance. The method also includes determining a first ICP value based on at least the information associated the first acoustic reflectance. Furthermore, the method includes measuring a second acoustic reflectance using the ear probe at a second time.

### CROSS-REFERENCES TO RELATED APPLICATIONS

This application claims priority from U.S. Provisional Application No. 60/788,984, filed Apr. 3, 2006, which is herein incorporated by reference for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention relates in general to medical diagnostic and monitoring techniques. More particularly, the invention provides a method and system for monitoring intracranial pressure. In a specific embodiment, the present invention provides a method and system for non-intrusive monitoring of intracranial pressure using acoustic based instruments. Merely by way of example, the invention is described as it applies to medical diagnostics and monitoring, but it should be recognized that the invention has a broader range of applicability.

Measuring and/or continuous monitoring of intracranial pressure (ICP) are important aspects of diagnosing treating head injuries and/or other conditions. For example, a change in ICP may be an indication of brain tumor, meningitis, brain swelling, increase venous pressure, etc. ICP is typically defined as the pressure exerted by the cranium on the brain tissue, cerebrospinal fluid, and the brain's circulating blood volume. Usually, when amount of various fluids within a cranium increases (e.g., caused by swelling from head injuries, or others), the ICP increases, as the cranium is characterized by a fixed volume.

To properly treat various head injuries, it is often important to continuously monitor the ICP of patients. Increase of ICP from the normal level may cause brain trauma and other serious conditions. With ICP being continuously monitored, it is then possible to conduct proper treatments accordingly.

Over the past, monitoring ICP has been an invasive procedure. For example, conventional techniques for measuring CIP involves a direct entry of a probe system through the skull. These techniques often have undesirable side effects, namely damages to skulls and likelihood of infection from probe openings.

In the recent years, various non-invasive techniques have been developed. For example, one of the non-invasive techniques operates under the principle of distortion product otoacoustic emissions (DPOAEs). Unfortunately, these techniques are often inadequate for various purposes.

Therefore, it is desired to have novel and improved techniques for monitoring and measuring ICP.

### BRIEF SUMMARY OF THE INVENTION

The present invention relates in general to medical diagnostic and monitoring techniques. More particularly, the invention provides a method and system for monitoring intracranial pressure. In a specific embodiment, the present invention provides a method and system for non-intrusive monitoring of intracranial pressure using acoustic based instruments. Merely by way of example, the invention is described as it applies to medical diagnostics and monitoring, but it should be recognized that the invention has a broader range of applicability.

According to an embodiment, the present invention provides a method for monitoring intracranial pressure (ICP) for at least one patient. The method includes positioning an ear probe into an ear canal. The method further includes measuring a first acoustic reflectance using the ear probe at a first time. The first acoustic reflectance is associated with the ear canal as a function of an incident pressure and an acoustic frequency. The method additionally includes processing information associated with the first acoustic reflectance. The method also includes determining a first ICP value based on at least the information associated the first acoustic reflectance. Furthermore, the method includes measuring a second acoustic reflectance using the ear probe at a second time. The method also includes processing information associated with the second acoustic reflectance. The method further includes determining a second ICP value based on at least the information associated the second acoustic reflectance. Moreover, the method includes determining a status for the patient based on a relationship between the first ICP value and the second ICP value.

According to another embodiment, the presenting invention provides a method for measuring an intracranial pressure (ICP) value for a patient. The method includes positioning an ear probe into the patient's ear canal. The method further includes measuring a first acoustic reflectance using the ear probe at a first time. The first acoustic reflectance is associated with an ear canal as a function of an incident pressure and an acoustic frequency. The method further includes processing information associated with the first acoustic reflectance. The method also includes determined a parameter based on the first acoustic reflectance. The method additionally includes measuring a second acoustic reflectance using the ear probe at a second time. The method also includes determining an ICP value based on at least the information associated the second acoustic reflectance using the parameter.

According to yet another embodiment, the present invention provides a system for monitoring intracranial pressure (ICP) for at least one patient. The system includes a monitoring module configured to determining a change of ICP values. The ICP values is associated with the at least one patient. The system also includes an ear probe that is coupled to the monitoring module and configured to generate signals within an ear cavity of the at least one patient. The system further includes a display configured for displaying information associated with the ICP values. The ear probe is configured to generate a first signal and measure a first acoustic reflectance at a first time. The first acoustic reflectance is associated with an ear canal as a function of the first signal. The monitoring module is configured to process information associated the first acoustic reflectance, to determining a first ICP value based on at least the information associated the first acoustic reflectance. The ear probe is further configured to generate a second signal and to measure a second acoustic reflectance at a second time. The monitoring module is further configured process information associated the second acoustic reflectance, to determine a second ICP value based on at least the information associated the second acoustic reflectance, and to determine a status for the patient based on a relationship between the first ICP value and the second ICP value.

According to yet another embodiment, the present invention provides a method for monitoring intracranial pressure (ICP) for at least one patient. The method includes positioning an ear probe into an ear canal. The method also includes measuring a first middle ear power value using the ear probe at a first time. The first middle ear power value is associated with the ear canal as a function of an incident pressure and an acoustic frequency. The method further includes processing information associated with the first middle ear power value. The method additionally includes determining a first ICP value based on at least the information associated the first middle ear power value. The method further includes measuring a second middle ear power value using the ear probe at a second time. The method also includes processing information associated with the second middle ear power value. Moreover, the method includes determining a second ICP value based on at least the information associated the second middle ear power value.

According to yet another embodiment, the present invention provides a method for monitoring intracranial pressure (ICP) for at least one patient. The method includes positioning an ear probe into an ear canal. The method further includes providing a protocol for monitoring an ICP value. The method further includes measuring a first middle ear power value using the ear probe at a first time. The first middle ear power value is associated with the ear canal as a function of an incident pressure and an acoustic frequency. The first middle ear power value is associated with the ICP value. The method also includes processing information associated with the first middle ear power value. The method further includes measuring a second middle ear power value using the ear probe at a second time. The method additionally includes processing information associated with the second middle ear power value. The method further includes determining a relationship between the first middle ear power value and the second power value. The method also includes providing an indication based on the relationship.

According to yet another embodiment, the present invention provides a method for monitoring intracranial pressure (ICP) for at least one patient. The method includes positioning an ear probe into an ear canal. The method further includes providing a protocol for monitoring an ICP value. The method additionally includes measuring a first middle ear power value using the ear probe at a first time. The first middle ear power value is associated with the ear canal as a function of an incident pressure and an acoustic frequency. The first middle ear power value is associated with the ICP value. The method additionally includes processing information associated with the first middle ear power value. The method includes measuring a second middle ear power value using the ear probe at a second time. The method also includes processing information associated with the second middle ear power value. The method further includes determining a relationship between the first middle ear power value and the second power value. The method also includes providing an indication based on the relationship.

It is to be appreciated that the embodiments of the present invention provides various advantage over conventional techniques. In various embodiments, the present invention provides an non-intrusive technique for determining and monitoring ICP. In addition, embodiments of the present invention are useful for many types of patients, especially those who have hearing problems. In certain embodiments, the present invention is implemented in conjunction with conventional techniques. Also, embodiments of the present invention are compatible with conventional systems and techniques. There are other benefits as well.

Depending upon embodiment, one or more of these benefits may be achieved. These benefits and various additional objects, features and advantages of the present invention can be fully appreciated with reference to the detailed description and accompanying drawings that follow.

### BRIEF DESCRIPTION OF THE DRAWINGS

FIG. 1 is a simplified diagram of an ICP monitoring system according to an embodiment of the present invention.

FIG. 2 is a simplified diagram illustrating a process for monitoring the ICP according to an embodiment of the present invention.

FIG. 3 is a simplified diagram illustrating graphs for monitoring ICP fluctuations according to an embodiments of the present invention.

FIG. 4 is a simplified diagram illustrating a system for monitoring multiple patients according to an embodiment of the present invention.

### DETAILED DESCRIPTION OF THE INVENTION

The present invention relates in general to medical diagnostic and monitoring techniques. More particularly, the invention provides a method and system for monitoring intracranial pressure. In a specific embodiment, the present invention provides a method and system for non-intrusive monitoring of intracranial pressure using acoustic based instruments. Merely by way of example, the invention is described as it applies to medical diagnostics and monitoring, but it should be recognized that the invention has a broader range of applicability.

As described above, various conventional non-invasive techniques have been developed for measuring and monitoring ICP. For example, the DPOAE technique, which has traditionally been used for diagnosing hearing problems, has recently been adopted for determining ICP.

The measurement of otoacoustic emissions provides an non-invasive means of measuring auditory function. Otoacoustic emissions (OAEs) are involuntary sounds generated by the outer hair cells (OHCs) within the cochlea, either spontaneously or in response to a stimulus. There are various types of OAE (e.g., spontaneous emissions, evoked otoacoustic emissions, etc.). Spontaneous otoacoustic emissions (SOAEs) occur in the absence of external stimulation while evoked otoacoustic emissions (EOAEs) occur during or after external acoustic stimulation. Transient otoacoustic emissions (TOAE) are evoked by short impulsive sounds. Distortion product otoacoustic emissions (DPOAE) are evoked by pairs of tones. For example, tonal stimuli at frequencies of f1 and f2 will evoke an otoacoustic emission at a frequency of 2f2-f1. Since the frequency of the emission is known, it is possible to extract the signal from background noise with a high degree of accuracy even though the level of the evoked otoacoustic emission is relatively low.

Over the past, researches have been done with OAE based ICP measurements. For example, several studies have shown that evoked otoacoustic emissions (OAEs) are sensitive to changes in ICP. That is, changes in ICP produce changes in the sound transmission characteristics of the inner and middle ear which cause changes of the amplitude of the evoked otoacoustic emissions. Typically, evoked OAEs are influenced by middle ear transmissions in both the forward direction as the stimulus is transmitted to the cochlea and on its return as emission from the cochlea to the middle ear.

The use of OAE based techniques has been, in large part, useful in monitoring ICP changes on patients. Unfortunately, these techniques are often inadequate, especially for certain populations. For example, OAE based techniques is largely based on the inner hearing response from a patients hear before ICP measurements can be performed. For people with hearing problem or deaf, OAE based techniques cannot be used.

It is to be appreciated that various embodiments according the present invention are useful for many types of patients, including those whose ICP cannot be determined by the OAE based techniques. In contrast, embodiments of the present invention are useful for non-intrusively measuring and monitoring ICP for almost all types of patients. As explained above, various embodiments of the present invention are useful for a wide range of diagnosis that are related to ICP. For example, application includes, in addition to monitoring head injuries, diagnosing strokes, hydrocephalus, brain tumor, brain injury, CSF leak, etc. In addition, applications of the present invention also includes performing measurements in preparation for brain surgery. There are many other applications as well. Certain principles according to the present invention is described in U.S. application Ser. No. 11/061,368, filed Feb. 18, 2005, which is herein incorporated by reference.

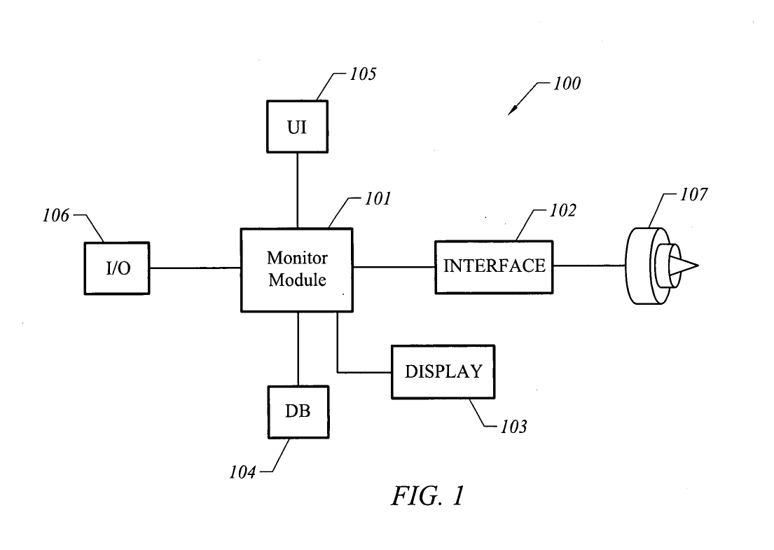

FIG. 1 is a simplified diagram of an ICP monitoring system according to an embodiment of the present invention. This diagram is merely an example, which should not unduly limit the scope of the claims. One of ordinary skill in the art would recognize many variations, alternatives, and modifications. As shown in FIG. 1, an ICP monitoring system 100 includes the following components:
1. a monitor module 101;
2. an interface 102;
3. a display 103;
4. a database 104;
5. a user interface 105;
6. an I/O port 106; and
7. an ear probe 107.

It is to be understood that the system 100 merely provides an illustration. According to various embodiments, various components may be added or removed as contemplated by the present invention.

The module 101 is configured to perform a variety of functions associated monitoring ICP. In addition to monitoring, the module 101 is also capable of perform detailed measurement of ICP. According to a specific embodiment, the module 101 is implemented by a special purpose apparatus that is manufactured for the sole purpose of monitoring ICP. According to certain embodiments, the module 101 may be implemented by general purpose devices, such as personal computers and handheld devices. As an example, the module 101 is implemented with a personal digital assistant that is portable for a variety of applications.

In a specific embodiment, the module 101 provides control signals for performing reflectance measurements. For example, the module 101 generates signals at various frequencies for determining reflectance. As another example, the module 101 determines reflectance values based on various signals values. Using the reflectance values, the module 101 determines and/or compare ICP values. For example, the module 101 determines ICP values and produces graphical representation of changes in ICP values at the display 103. According to various embodiments, the display 103 could be a CRT monitor, an LCD display, and touch sensitive screen, and others. The display 103 may provides other information in addition to ICP values. For example, the display 103 may provide warning signs in certain color schemes (e.g., red, yellow, etc.) when ICP values change greatly.

The module 101, as shown in FIG. 1, is connected to various components and/or peripherals. Depending on the application, these components and/or peripherals may internal components of the module 101.

The interface 102 is used to provide a connection between the monitor module 101 and the ear probe 107. According to certain embodiments, the interface 102 connects to the module 101 via a USB port and connects to the probe 107 via a 7 pin DIN connector, thereby allowing the monitor module 101 to be connected to the probe 107. In a specific embodiment, the interface 102 includes protection circuit for shielding the signals from unwanted noises and/or interferences. Depending on the application, the interface 102 may include other types of connector and/or adapters. For example, the interface 102 includes a PCMCIA card interface for interfacing with an notebook computer.

The ear probe 107 is shaped to be positioned within ear canals (or cavity) and is configured to produce sound waves (which could be audible or inaudible) and to sense response for the sound waves. According to an embodiment, the ear probe 107 includes probe speakers. When the probe 107 receives a signals (e.g., acoustic signals for performing diagnosis) from the module 101 via the interface 102, the probe speakers produces the sound wave within the inserted ear canal. The probe 107 is further configure to collect various measures. For example, the probe 107 is able to detect and measure incident pressure and reflect pressure from the ear canal (e.g., eardrum and the cochlea). The measured values are then sent to the module 101 via the interface 102. Depending on the application, the ear probe 107 may be configured to generate sound waves at different frequencies (e.g., for performing DPOAE related measurements).

In various embodiments, the system 100 is configured to work with a variety of devices. For example, the system 100 includes an I/O port 106, which may be in various configurations for connectivity with a variety of apparatus and peripherals. For example, the system 100 is connected to network module via the I/O port 106, which allows the system 100 to send data to other devices (e.g., a doctor or nurse's pager for notification). As another example, the I/O port 106 allows the systems to connect to other types of medical monitoring devices.

According to a specific embodiment, the module 101 is connected to the database 104. It is to be understood that the database 104 maybe implemented as a part of the module 101. The database 104 includes various information related to monitoring and/or measuring ICP. For example, the database includes information that is specific to a patent whose ICP is to be measured and/or monitored. Such information may include previous ICP measures, and various parameters (e.g., default reflectance value, calibration value, etc.) that are specific to the patient. As another example, the database includes various protocols for performing measurements. These protocols include specific measurements for testing to be performed. For example, for serious head injuries, the protocol dictates that series ICP measurements to be performed in short time intervals. For certain diagnostic measurements, the ICP measurement is performed in a high degree of accuracy.

In a specific embodiments, the database 104 includes information associated with specific monitoring protocols. For example, during a monitoring process, if ICP values fluctuates more than a threshold value that is stored in the database 104, the database 104 provides a status indication that is associated with the fluctuation.

In addition to providing information for performing measurements, the database 104 can also be used to store information associated with ICP measurements. In certain embodiments, the database 104 is implemented using a hard drive. Depending on the application, the database may be implemented using other types of storage devices, such as a network drive, flash memories, etc.

The module 101 is further connected to the user interface 105. In certain embodiments, the user interface 105 may be a keyboard, a mouse, and/or a touch screen. Through the user interface 105, an operator of the system 100 is able to adjust various parameters for monitoring and/or measuring ICP, and also to performing various measurements.

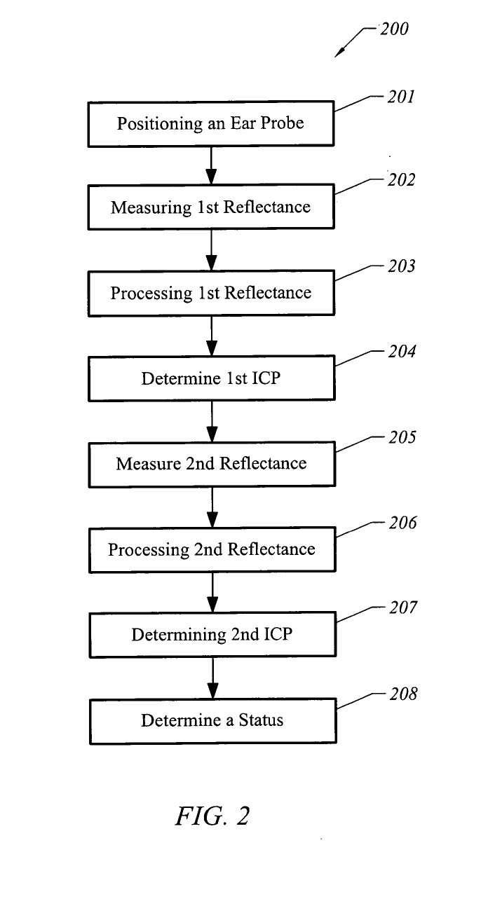

FIG. 2 is a simplified diagram illustrating a process for monitoring the ICP according to an embodiment of the present invention. This diagram is merely an example, which should not unduly limit the scope of the claims. One of ordinary skill in the art would recognize many variations, alternatives, and modifications. For example, various steps as described below may be added, removed, replaced, rearranged, repeated, overlapped, and/or partially overlapped. A process 200 includes the following steps:
step 201: positioning an ear probe into the patient's ear canal
step 202: measuring a first acoustic reflectance using the ear probe at a first time;
step 203: processing information associated with the first acoustic reflectance;
step 204: determining a first ICP value based on at least the information associated the first acoustic reflectance;
step 205: measuring a second acoustic reflectance using the ear probe at a second time;
step 206: processing information associated with the second acoustic reflectance;
step 207: determining a second ICP value based on at least the information associated the second acoustic reflectance
step 208: determining a status for the patient based on a relationship between the first ICP value and the second ICP value.

At step 201, an ear probe is positioned into an patient ear. In certain embodiments, an ear probe of appropriate size is selected based on a patient's ear size. For example, the ear probe is shaped to be positioned within ear canals (or cavity) and is configured to produce sound waves (which could be audible or inaudible) and to sense response for the sound waves. According to an embodiment, the ear probe includes probe speakers. When the probe receives a signals (e.g., acoustic signals for performing diagnosis) from the module via the interface, the probe speakers produces the sound wave within the inserted ear canal. The probe is further configure to collect various measures. For example, the probe is able to detect and measure incident pressure and reflect pressure from the ear canal (e.g., eardrum and the cochlea). Depending on the application, the ear probe may be configured to generate sound waves at different frequencies (e.g., for performing DPOAE related measurements).

At step 202, a reflectance value is measured. As explained above, reflectance value is a function of incidence pressure and reflected pressured within the ear cavity. For example, the ear probe produces a signal at predetermined frequency and pressure levels. Next, the ear probe measured the frequency and pressure level of the reflected signal from the middle ear of the ear cavity. Based on the predetermined levels and the measured levels, a reflectance value is generated. It is to be appreciated that other measurements (e.g., power reflectance, power transmittance, resistance, conductance, etc.) associated with reflectance can be used for monitoring ICP as well.

In certain embodiments, calibration is performed prior to the reflectance measurement. For example, during the calibration processes the ear probe produces and measure signals at predetermined frequencies and levels to ensure that proper measurement can be obtained.

At step 203, the measure reflectance value is processed. According to an embodiment, noise filtering is performed to ensure that the reflectance value is accurate. For example, the noise filtering process removes various undesirable noise that might come from a variety of sources, such as ambience noise, electrical noise, etc. In some embodiments, measurements related to the reflectance value are generated.

At step 203, an ICP value is determined based on the reflectance value. According to certain embodiments, a relationship between ICP values and reflectance values is predetermined. For example, the ICP value is determined by looking up a table for corresponding reflectance value. According to a specific embodiment, a relationship between ICP value and reflectance value is specific to the patient and stored in a database. By looking up to the database, an ICP value is determined. In some embodiments, the ICP value is determined using both reflectance and DPOAE measurements.

At step 205, a measurement is performed to determine the reflectance at a time after step 202. For example, the measurement performed at step 205 is to monitor changes in ICP for the patient. In a specific embodiment, the new measurement is performed every 2 minutes. Depending on the application, the frequency at which measurement is performed vanes.

At step 206, the measure reflectance value is processed. According to an embodiment, noise filtering is performed to ensure that the reflectance value is accurate. For example, the noise filtering process removes various undesirable noise that might come from a variety of sources, such as ambience noise, electrical noise, etc. In some embodiments, measurements related to the reflectance value are generated.

At step 207, an ICP value is determined based on the reflectance value. According to certain embodiments, a relationship between ICP values and reflectance values is predetermined. For example, the ICP value is determined by looking up a table for corresponding reflectance value. According to a specific embodiment, a relationship between ICP value and reflectance value is specific to the patient and stored in a database. By looking up to the database, an ICP value is determined. In some embodiments, the ICP value is determined using both reflectance and DPOAE measurements.

At step 208, a status is determined based on the ICP values measured at the first and the second time. In a specific embodiment, a warning indication (e.g., warning sound, red light, etc.) is generated if it is determined that the ICP value has change by a significant amount. For example, if the ICP value has increased by a predetermined amount or percentage during a time interval, a warning indication is generated. In some embodiments, a warning indication is generated if the ICP value exceeds certain predetermined threshold value. Depending on the application, the criteria for providing a warning indication may be based on policies stored in a database.

It is to be appreciated that the process 200 described above is useful for performing various types of measurements and/or monitoring. For example, the process 200 is used to continuously monitor one or more patients' ICP reading.

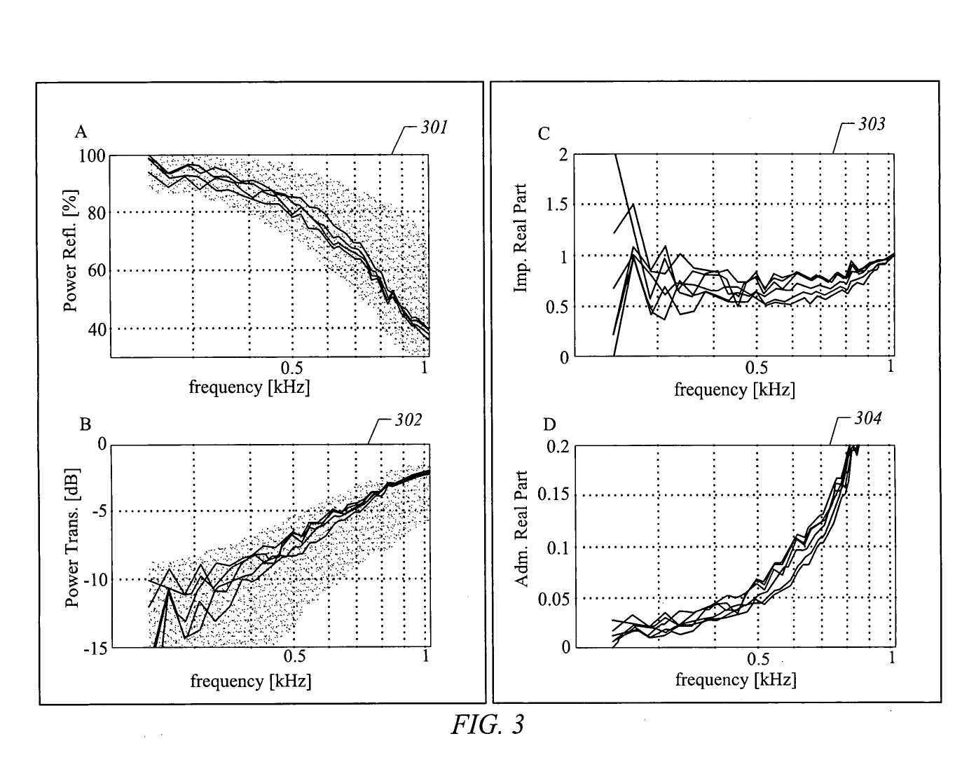

FIG. 3 is a simplified diagram illustrating graphs for monitoring ICP fluctuations according to an embodiments of the present invention. This diagram is merely an example, which should not unduly limit the scope of the claims. One of ordinary skill in the art would recognize many variations, alternatives, and modifications. The plot 301 illustrates a relationship between reflectance and frequency. The plot 302 illustrates a relationship between transmittance and frequency. The plot 303 illustrates a relationship between impedance and frequency. The plot 304 illustrates a relationship between conductance and frequency. As can be seen from these plots, changes in the values of these measurements are associate with the ICP and therefore can be used direct and/or indirectly as an indication for the ICP.

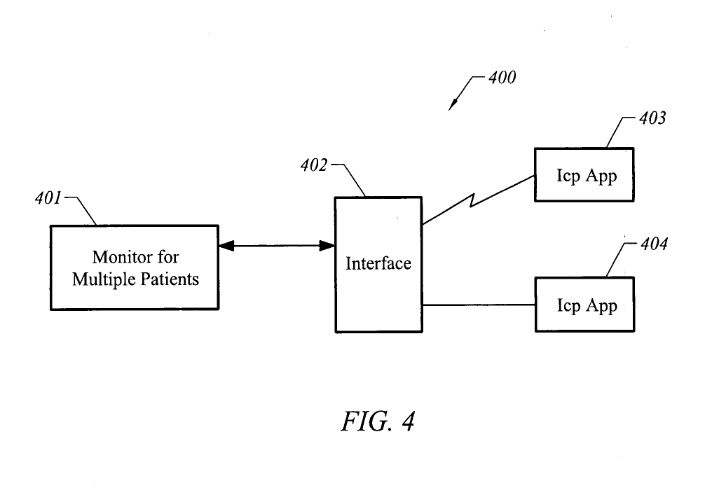

FIG. 4 is a simplified diagram illustrating a system for monitoring multiple patients according to an embodiment of the present invention. This diagram is merely an example, which should not unduly limit the scope of the claims. One of ordinary skill in the art would recognize many variations, alternatives, and modifications. As shown in FIG. 4, a system 400 includes a monitor 401, which is connected to units 403 and 404 via an interface 402. According to various embodiments, the units monitoring apparatus for measuring and/or monitoring patients one at a time. Merely as an example, the unit 403 is the system 100 illustrated according to FIG. 1. For example, the monitor 401 is connected to the unit 403 via a wireless network connection through the interface 402 and connected to the unit 404 via a wired connection. For example, the monitor 401 is provided for monitoring ICPs for one or more patients at using different units. In certain settings, the system 400 is used in a hospital emergency room for monitoring ICP for many patients at once.

According to an embodiment, the present invention provides a method for monitoring intracranial pressure (ICP) for at least one patient. The method includes positioning an ear probe into an ear canal. The method further includes measuring a first acoustic reflectance using the ear probe at a first time. The first acoustic reflectance is associated with the ear canal as a function of an incident pressure and an acoustic frequency. The method additionally includes processing information associated with the first acoustic reflectance. The method also includes determining a first ICP value based on at least the information associated the first acoustic reflectance. Furthermore, the method includes measuring a second acoustic reflectance using the ear probe at a second time. The method also includes processing information associated with the second acoustic reflectance. The method further includes determining a second ICP value based on at least the information associated the second acoustic reflectance. Moreover, the method includes determining a status for the patient based on a relationship between the first ICP value and the second ICP value. For example, the embodiment is illustrated according to FIG. 2.

According to another embodiment, the presenting invention provides a method for measuring an intracranial pressure (ICP) value for a patient. The method includes positioning an ear probe into the patient's ear canal. The method further includes measuring a first acoustic reflectance using the ear probe at a first time. The first acoustic reflectance is associated with an ear canal as a function of an incident pressure and an acoustic frequency. The method further includes processing information associated with the first acoustic reflectance. The method also includes determined a parameter based on the first acoustic reflectance. The method additionally includes measuring a second acoustic reflectance using the ear probe at a second time. The method also includes determining an ICP value based on at least the information associated the second acoustic reflectance using the parameter. For example, the embodiment is illustrated according to FIG. 2.

According to yet another embodiment, the present invention provides a system for monitoring intracranial pressure (ICP) for at least one patient. The system includes a monitoring module configured to determining a change of ICP values. The ICP values is associated with the at least one patient. The system also includes an ear probe that is coupled to the monitoring module and configured to generate signals within an ear cavity of the at least one patient. The system further includes a display configured for displaying information associated with the ICP values. The ear probe is configured to generate a first signal and measure a first acoustic reflectance at a first time. The first acoustic reflectance is associated with an ear canal as a function of the first signal. The monitoring module is configured to process information associated the first acoustic reflectance, to determining a first ICP value based on at least the information associated the first acoustic reflectance. The ear probe is further configured to generate a second signal and to measure a second acoustic reflectance at a second time. The monitoring module is further configured process information associated the second acoustic reflectance, to determine a second ICP value based on at least the information associated the second acoustic reflectance, and to determine a status for the patient based on a relationship between the first ICP value and the second ICP value. For example, the embodiment is illustrated according to FIG. 1.

According to yet another embodiment, the present invention provides a method for monitoring intracranial pressure (ICP) for at least one patient. The method includes positioning an ear probe into an ear canal. The method also includes measuring a first middle ear power value using the ear probe at a first time. The first middle ear power value is associated with the ear canal as a function of an incident pressure and an acoustic frequency. The method further includes processing information associated with the first middle ear power value. The method additionally includes determining a first ICP value based on at least the information associated the first middle ear power value. The method further includes measuring a second middle ear power value using the ear probe at a second time. The method also includes processing information associated with the second middle ear power value. Moreover, the method includes determining a second ICP value based on at least the information associated the second middle ear power value. For example, the embodiment is illustrated according to FIG. 2.

According to yet another embodiment, the present invention provides a method for monitoring intracranial pressure (ICP) for at least one patient. The method includes positioning an ear probe into an ear canal. The method further includes providing a protocol for monitoring an ICP value. The method further includes measuring a first middle ear power value using the ear probe at a first time. The first middle ear power value is associated with the ear canal as a function of an incident pressure and an acoustic frequency. The first middle ear power value is associated with the ICP value. The method also includes processing information associated with the first middle ear power value. The method further includes measuring a second middle ear power value using the ear probe at a second time. The method additionally includes processing information associated with the second middle ear power value. The method further includes determining a relationship between the first middle ear power value and the second power value. The method also includes providing an indication based on the relationship. For example, the embodiment is illustrated according to FIG. 2.

According to yet another embodiment, the present invention provides a method for monitoring intracranial pressure (ICP) for at least one patient. The method includes positioning an ear probe into an ear canal. The method further includes providing a protocol for monitoring an ICP value. The method additionally includes measuring a first middle ear power value using the ear probe at a first time. The first middle ear power value is associated with the ear canal as a function of an incident pressure and an acoustic frequency. The first middle ear power value is associated with the ICP value. The method additionally includes processing information associated with the first middle ear power value. The method includes measuring a second middle ear power value using the ear probe at a second time. The method also includes processing information associated with the second middle ear power value. The method further includes determining a relationship between the first middle ear power value and the second power value. The method also includes providing an indication based on the relationship. For example, the embodiment is illustrated according to FIG. 2.

It is to be appreciated that the embodiments of the present invention provides various advantage over conventional techniques. In various embodiments, the present invention provides an non-intrusive technique for determining and monitoring ICP. In addition, embodiments of the present invention are useful for many types of patients, especially those who have hearing problems. In certain embodiments, the present invention is implemented in conjunction with conventional techniques. Also, embodiments of the present invention are compatible with conventional systems and techniques. There are other benefits as well.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A method for monitoring intracranial pressure (ICP) for at least one patient, the method comprising:
positioning an ear probe into an ear canal;
measuring a first acoustic reflectance using the ear probe at a first time, the first acoustic reflectance being associated with the ear canal as a function of an incident pressure and an acoustic frequency;
processing information associated with the first acoustic reflectance;
determining a first ICP value based on at least the information associated the first acoustic reflectance;
measuring a second acoustic reflectance using the ear probe at a second time;
processing information associated with the second acoustic reflectance;
determining a second ICP value based on at least the information associated the second acoustic reflectance; and
determining a status for the patient based on a relationship between the first ICP value and the second ICP value.

2. The method of claim 1 further comprising choosing the ear probe, the ear probe being associated a size of the ear canal.

3. The method of claim 1 further comprising obtaining a profile associated with the at least one patient.

4. The method of claim 1 further comprising generating a warning signal if the first ICP value is greater than the second ICP value by a predetermined amount.

5. The method of claim 1 further comprising generating a warning signal if the second ICP value is greater 110 percent than the first ICP value.

6. The method of claim 1 wherein the measuring a first acoustic reflectance comprises determining an ratio between a incident pressure and a reflectance pressure.

7. The method of claim 1 further comprising:
performing operations to reduce ICP if the second ICP value is greater than the first ICP value by a predetermined amount.

8. The method of claim 1 wherein the second time is approximately five minutes after the first time.

9. A method for measuring an intracranial pressure (ICP) value for a patient, the method comprising:
positioning an ear probe into the patient's ear canal;
measuring a first acoustic reflectance using the ear probe at a first time, the first acoustic reflectance being associated with an ear canal as a function of an incident pressure and an acoustic frequency;
processing information associated with the first acoustic reflectance;
determined a parameter based on the first acoustic reflectance;
measuring a second acoustic reflectance using the ear probe at a second time; and
determining an ICP value based on at least the information associated the second acoustic reflectance using the parameter.

10. The method of claim 9 further comprising generating a warning signal if the ICP value is greater than a predetermined level.

11. The method of claim 9 wherein the second acoustic reflectance is measured within a range of approximately 200 to 1000 Hz.

12. The method of claim 9 further comprising diagnosing a likelihood of stroke based on the ICP value.

13. The method of claim 9 further comprising diagnosing a likelihood of brain tumor based on the ICP value.

14. The method of claim 9 further comprising diagnosing a likelihood of CSF leak based on the ICP value.

15. The method of claim 9 further comprising preparing for a brain surgery.

16. A system for monitoring intracranial pressure (ICP) for at least one patient, the system comprising:
a monitoring module configured to determining a change of ICP values, the ICP values being associated with the at least one patient;
an ear probe, the ear probe being coupled to the monitoring module and configured to generate signals within an ear cavity of the at least one patient;
a display configured for displaying information associated with the ICP values;
wherein:
the ear probe is configured to generate a first signal and measure a first acoustic reflectance at a first time, the first acoustic reflectance being associated with an ear canal as a function of the first signal;
the monitoring module is configured to process information associated the first acoustic reflectance, to determining a first ICP value based on at least the information associated the first acoustic reflectance;
the ear probe is further configured to generate a second signal and to measure a second acoustic reflectance at a second time;
the monitoring module is further configured process information associated the second acoustic reflectance, to determine a second ICP value based on at least the information associated the second acoustic reflectance, and to determine a status for the patient based on a relationship between the first ICP value and the second ICP value.

17. The system of claim 16 wherein the monitoring module comprises a personal digital assistant (PDA).

18. The system of claim 16 wherein the monitoring module comprises a laptop computer.

19. The system of claim 16 wherein further comprising a display, the display being configured to show the relationship between the first ICP value and the second ICP value.

20. The system of claim 16 wherein further comprising a display, the display being configured to displace the status.

21. The system of claim 16 wherein further comprising a speaker, the speaker being configured to generate a warning sound based on the status.

22. The system of claim 16 further comprising an interface module, the interface module being coupled to the ear probe and the monitoring module.

23. The system of claim 16 wherein the ear probe comprises:
a speaker for generate the first and the second signals;
a sensor for measure the first and the second acoustic reflectance.

24. A method for monitoring intracranial pressure (ICP) for at least one patient, the method comprising:
positioning an ear probe into an ear canal;
measuring a first middle ear power value using the ear probe at a first time, the first middle ear power value being associated with the ear canal as a function of an incident pressure and an acoustic frequency;
processing information associated with the first middle ear power value;
determining a first ICP value based on at least the information associated the first middle ear power value;
measuring a second middle ear power value using the ear probe at a second time;
processing information associated with the second middle ear power value; and
determining a second ICP value based on at least the information associated the second middle ear power value.

25. The method of claim 24 further comprising determining a third ICP value based on a octoacoustic emission value.

26. The method of claim 24 wherein the processing information associated the first middle ear power value comprises removing noises.

27. The method of claim 24 wherein the first middle ear power value comprises a reflectance value.

28. The method of claim 24 wherein the first middle ear power value comprises a transmittance value.

29. The method of claim 24 wherein the first middle ear power value comprises a resistance value.

30. The method of claim 24 wherein the first middle ear power value comprises a conductance value.

31. A method for monitoring intracranial pressure (ICP) for at least one patient, the method comprising:
positioning an ear probe into an ear canal;
providing a protocol for monitoring an ICP value;
measuring a first middle ear power value using the ear probe at a first time, the first middle ear power value being associated with the ear canal as a function of an incident pressure and an acoustic frequency, the first middle ear power value being associated with the ICP value;
processing information associated with the first middle ear power value;
measuring a second middle ear power value using the ear probe at a second time;
processing information associated with the second middle ear power value; and
determining a relationship between the first middle ear power value and the second power value; and
providing an indication based on the relationship.

32. The method of claim 31 wherein the indication comprises a plot.

33. The method of claim 31 wherein the indication comprises a warning signal.

34. The method of claim 31 wherein the indication comprises a warning sound.

35. The method of claim 31 wherein the first middle ear power value comprises a reflectance value.

36. The method of claim 31 wherein the first middle ear power value comprises a transmittance value.
